(19) United States
(12) Patent Application Publication
(10) Pub. No.: US 2026/0179774 A1
(43) Pub. Date: Jun. 25, 2026

(54) Artificial intelligence based system for generating personalized medical information

(71) Applicants: János Rokai, Budapest (HU); Gergely Márton, Budapest (HU)

(72) Inventors: János Rokai, Budapest (HU); Gergely Márton, Budapest (HU)

(21) Appl. No.: 19/427,511

(22) Filed: Dec. 19, 2025

Related U.S. Application Data
(60) Provisional application No. 63/736,022, filed on Dec. 19, 2024.

(51) Int. Cl. G16H 50/20

(52) U.S. Cl. CPC G16H 50/20

(57) ABSTRACT

The invention provides an artificial intelligence-based system for generating personalized medical information through the integration of multi-modal data across pre-hospitalization, hospitalization, and post-hospitalization phases. The system employs encoder modules to process diverse input modalities, including audio recordings, video streams, biomedical images, text-based clinical records, and physiological signals. These encoded representations are integrated into a unified latent space using a large language model (LLM) trained on medical datasets comprising historical patient cases, anatomical knowledge, and treatment guidelines. The LLM enables cross-modal analysis to generate personalized outputs via decoder modules, which transform the latent space representation into actionable formats like text-based summaries, visualizations, audio explanations, and treatment suggestions. A key innovation is real-time intraoperative feedback via encoder-decoder modules detecting anatomical structures and deviations from standard protocols. The system also includes a federated learning module to aggregate model updates across medical centers while preserving patient privacy through deidentification protocols.

### BACKGROUND OF THE INVENTION

Modern medical care is increasingly data-intensive, requiring healthcare professionals to process and interpret vast volumes of patient information across multiple stages pre-hospitalization, hospitalization, and post-hospitalization. This complexity arises from the integration of diverse modalities such as clinical notes, diagnostic imaging, physiological signals, audio-visual documentation (e.g., surgical videos), and genomic data. While electronic health records (EHRs) and telemedicine platforms have improved access to patient information, they often remain siloed systems that fail to provide a unified, real-time view of the patient's condition or treatment trajectory.

The quality of medical care is significantly influenced by the experience and expertise of individual healthcare providers. In large urban hospitals with high patient volumes, clinicians are more likely to encounter rare or complex cases, thereby gaining specialized knowledge over time. Conversely, in smaller clinics or remote areas, limited exposure to such cases can lead to variability in diagnostic accuracy and treatment decisions. Although digital medical databases and research repositories have expanded access to clinical guidelines and evidence-based practices, the practical application of this knowledge remains unevenly distributed across healthcare settings.

National and international medical guidelines aim to standardize care protocols; however, these are typically generalized and do not account for individual patient variability or contextual factors such as comorbidities, genetic predispositions, or lifestyle. As a result, clinical decisions often rely heavily on the subjective experience of the treating physician rather than an objective synthesis of all available data.

The emergence of novel diseases, coupled with limited exposure in certain medical centers, increases the risk of misdiagnosis and suboptimal treatment planning. Additionally, the rapid development of new pharmaceuticals necessitates continuous education for healthcare professionals to stay informed about evolving therapeutic options, drug interactions, and potential side effects. Failure to do so can compromise patient safety.

Post-treatment, patients frequently have unresolved questions or concerns regarding their condition, prognosis, and prescribed therapies. Due to time constraints during clinical visits, medical staff may not address all queries in sufficient detail, leaving patients to seek information from unverified online sources. This scenario increases the risk of exposure to misinformation, vague explanations, or conflicting advice.

Moreover, both patients and their families often struggle with retaining critical health-related information discussed during consultations. The absence of a structured, accessible interface for revisiting this information can lead to misunderstandings or missed follow-up actions-potentially endangering patient outcomes.

To address these challenges, various technologies have been developed, including EHR systems, telemedicine platforms, and AI-based diagnostic tools. However, existing solutions often operate in isolation, focusing on specific aspects of care without integrating data across modalities or stages of treatment. Furthermore, many current AI-driven medical systems suffer from limitations such as:
Narrow datasets: Reliance on limited or homogeneous training data that may not generalize well to diverse patient populations.
Lack of real-time adaptability: Inability to provide immediate feedback during critical clinical events (e.g., intraoperative procedures).
Inadequate personalization: Failure to synthesize multi-modal, longitudinal data into actionable insights tailored to the individual patient.

Recent advancements in artificial intelligence (AI), machine learning (ML), and natural language processing (NLP) have opened new possibilities for transforming medical care. AI systems can now analyze heterogeneous datasets, identify complex patterns, and generate predictive models that support clinical decision-making. However, existing implementations often lack a comprehensive architecture capable of integrating data across modalities, stages of treatment, and institutions.

The present invention addresses these shortcomings by introducing an artificial intelligence-based system for generating personalized medical information through the integration of multi-modal data using hierarchical deep learning architectures. The system employs encoder modules to process diverse input types—including audio recordings, video streams, biomedical images, text-based clinical records, and physiological signals—into a unified latent space representation. This representation is then analyzed by a large language model (LLM) trained on extensive medical datasets encompassing historical patient cases, anatomical knowledge, treatment guidelines, and pharmacological information.

A key innovation of the system lies in its ability to provide real-time intraoperative feedback during surgical procedures by analyzing live video streams and physiological data. Encoder modules detect anatomical structures and deviations from standard protocols, enabling immediate alerts or recommendations to assist surgeons in maintaining procedural accuracy and safety.

Additionally, the invention incorporates a federated learning module, allowing model updates to be aggregated across multiple medical centers without compromising patient privacy through de-identification techniques. This feature facilitates knowledge sharing between institutions while adhering to strict data protection standards.

The system also generates personalized outputs via decoder modules that transform latent space representations into actionable formats such as text-based summaries, visualizations, audio explanations, and treatment suggestions tailored to the individual patient's condition and medical history.

While several prior art systems have explored AI in healthcare (e.g., U.S. Pat. No. 10,496,788 B2; KR Patent No. 10-2558021 B1), none provide a comprehensive integration of multi-modal data across the entire patient care continuum or offer real-time feedback during critical clinical events. Similarly, existing literature (e.g., Moor et al., Nature, 2023) highlights the potential of foundation models in medical AI but does not address the specific challenges of cross-modal analysis and personalized output generation.

Therefore, there is a clear need for an integrated, intelligent system that leverages advanced machine learning techniques to provide personalized, real-time, and privacy-preserving support across all stages of patient care. The present invention fulfills this unmet need by offering a robust framework for synthesizing diverse medical data into actionable insights, thereby enhancing diagnostic accuracy, treatment personalization, and overall clinical efficiency.

Examples of relevant patents include:

| Patent no. | Patent title | Patent year | Patented in |
|---|---|---|---|
| KR102558021B1 | A clinical decision support | 2023 | KR |
| | ensemble system and the | | |
| | clinical decision support | | |
| | method by using the same | | |
| U.S. Pat. No. 10,496,788B2 | Holistic hospital patient | 2019 | US |
| | care and management | | |
| | system and method for | | |
| | automated patient | | |
| | monitoring | | |
| CN107615395B | Operating room black box | 2016 | US WO CA CN HK |
| | apparatus, system, method | | |
| | and computer readable | | |
| | medium for event and error | | |
| | prediction | | |
| U.S. Pat. No. 10,342,410B2 | Automated system for | 2017 | US |
| | medical video recording | | |
| | and storage | | |
| CN106709254B | A kind of medical diagnosis | 2016 | CN |
| | robot system | | |
| U.S. Pat. No. 8,271,415B2 | Computerized medical self- | 1993 | US |
| | diagnostic and treatment | | |
| | advice system including | | |
| | modified data structure | | |

Examples of relevant literature include:

| Paper title | Paper first author | Year | DOI |
|---|---|---|---|
| ChatGPT for healthcare | Mohd Javaid | 2023 | 10.1016/j.tbench.2023.100105 |
| services: An emerging stage | | | |
| for an innovative | | | |
| perspective | | | |
| Foundation models for | Michael Moor | 2023 | 10.1038/s41586-023- |
| generalist medical artificial | | | 05881-4 |
| intelligence | | | |
| Applications of Artificial | Z. Faizal khan | 2020 | 10.1155/2020/8894694 |
| Intelligence and Big Data | | | |
| Analytics in m-Health: A | | | |
| Healthcare System | | | |
| Perspective | | | |

### SUMMARY OF THE DISCLOSURE

The following terms are used consistently throughout this disclosure:

Tokenization: A process that converts subparts of input data (e.g., audio, text, images) into numerical vector representations. These vectors encode information about the subpart itself and its relationships with other subparts (e.g., positional or contextual properties).
Electronic Health Record (EHR): A digital repository containing a patient's medical history, diagnostic results, treatment plans, and other clinically relevant data.
Modality: Data types distinguished by their nature (e.g., audio, video, biomedical images, text) and characterized by unique properties defining their structure or origin.
Encoded Data: Data transformed via machine learning algorithms into a compressed representation that retains essential information for downstream processing.

The present invention handles the process of collection, analysis, and feedback of multi-modal data in a medical environment. The system consists of multiple hardware and software components connected through a secure network. The inputs of this multi-modal system are through various user interfaces, network interfaces, and sensory hardware. The system outputs information to various user interfaces, network interfaces, memory storage, and effector hardware. The system has an internal database and access to external, third-party databases. The system allows the recording and processing of information from multiple stages of patient hospitalization, as well as discrete and continuous data. The system allows for supervision at multiple points in the process of recording, analyzing, and generating feedback. The network connecting the different software and hardware components together is configured to allow real-time streaming of data in surgical operating rooms as well as for continuous analysis of the captured data, allowing real-time feedback/status updates on the different output components. The captured data and the processed information from other software components are also timestamped and stored in a database. The database can have parts with different levels of security and access policies to protect patient privacy. The system would have static and dynamic supervision. The static supervision could be made by an internal interface, with which the flow of information is supervised and/OR modified between the nodes as a constant factor. A dynamic supervision would be implemented in the system where the supervision is more personalized, per patient and the output of different nodes of software or hardware are manipulated by a supervisor on a case-by-case basis.

The system would entail several types of machine-learning algorithms to process information between nodes of the network. The network is capable of connecting to third-party databases and softwares. The connection and data transfer is made in a secure way and the data is deidentified. The incoming data to the system, are processed by machine learning methods, and the processed data is used to further process information of the same/OR of different kinds. The information analysis process will be an automated process with the aforementioned supervision after the different modalities are transformed into different knowledge vector spaces by other node(s) in the network. Software components of the system can be of single-/or multi-modality. The deep learning methods—implemented in certain nodes or software components—are pre-trained on medical data. Single-modality deep learning models are trained to internally encode incoming information as efficiently as possible and to output adequate information for the next node in the network. Multi-modal deep learning models are trained on multiple different modalities with preprocessed and/OR encoded and/OR raw data.

Hardware components of the system entail medical and non-medical devices as well. Medical hardware components connect to the system through standardized communication channels, either wired or wireless mode.

The system described here is embodied by a 3-part system: pre-hospitalization, hospitalization, and posthospitalization module. The main modules and submodules are by design multi-modal systems, while the accompanying encoder and decoder submodules analyze and capture single-modality data, from different sources with different outputs. The encoder, decoder, and central submodules can entail a wide range of complexity operations: from a single operation up to complex, billion-parameter deep learning models. In principle, encoders have as input raw-, or preprocessed data of a certain modality and transform the input data, which can be used as input to the central submodule or directly to the decoder. The central submodule in principle integrates data from different encoders and transforms the data in such a way that the decoders can operate on them. The decoders in principle are submodules that transform data into a specific modality.

The pre-hospitalization module collects, analyzes, categorizes, records, and integrates patient information that is generated before current hospitalization. The pre-hospitalization module has several encoder submodules, several decoder submodules, different input modalities, and multiple possible interaction interfaces. The information-recording process has several points where human supervision can be effectuated.

The hospitalization module has as its task to integrate the information from the pre-hospitalization module, generate suggestions for different examinations, and update the possible diagnosis suggestions, providing multiple possibilities. The hospitalization module is constructed of 2 parts: a preoperative part, and a surgical treatment part.

The post-hospitalization module has as its task to integrate the information from the pre-hospitalization module and hospitalization module, to compare the outputs of these modules to medical literature and to provide post-operation treatment suggestions, provide information on the hospitalization procedures for the patient, handle the registration and evaluation of follow-up information and provide further suggestions.

The encoders receive modality-specific inputs and transform the data into a more general latent space. A time-dependent or multi-dimensional data can be transformed into multiple vectors in the latent space. The input of the encoders can be supplied directly by the end user through a user interface or through a third-party recording device.

The decoders can receive input from the latent space in a specific order and they transform the received information into a modality-specific output. This output can be directly presented to the end-user by a user interface and/OR could be used as an input for a third-party effector.

The encoders and decoders are connected through a central submodule. This submodule transforms the information from the encoders into output data that will be used as input for the decoder modules.

The central submodule is responsible for integrating the different encoder submodules and for generating inputs for the decoder submodules. The central submodule creates an interactive interface both for the patient side and both for the medical professional staff side. The central submodule operates in various modes, such as automatic or semi-automatic models. In automatic mode, the central module uses the outputs of the encoders to decide on further interactive actions for the decoders. The fully automatic mode interacts with the patient in a fully automatic/unsupervised way. The semi-automatic mode does not have any direct interactivity possibility with the patient. The semi-automatic mode has two possible further modes: remote semi-automatic mode or onsite semi-automatic mode.

In some embodiments of the invention central multi-modal submodules are represented by Large Language Models (LLMs). The LLMs are trained on medical cases, with different focuses in the different phases. The LLMs are trained on previous studies, symptoms, theoretical background of general neuroscience, general anatomy like vascularization of the head and vertebrae, cranial and whole-body bone structure, and lymphatic system of head and vertebrae. The model can effectively perform suggestions in different subphases as well during the hospitalization of patients. The model has access to internal and external datasets as well. The internal datasets serve as an anchor point for local-specific disease parameters, while the external datasets serve as information for up-to-date information on guidelines, studies, and other medical sources.

### Prehospitalization Module

In some embodiment of the invention, a transcriber encoder module is implemented, that captures the consultation between medical staff and patients.

In some embodiment of the invention, a machine-learning-based encoder is used, to summarize patient “clinical history” and general information.

In some embodiment of the invention, a machine-learning-based encoder and/OR decoder are used to create case reports of the patients based on patient history, patient symptoms, and conversations with the medical staff.

In some embodiment of the invention, a decoder generates possible diagnosis options based on patient data and symptoms.

In some embodiment of the invention, an encoder-decoder module is implemented that has a user interface, with prewritten and/OR dynamically generated questions and multiple optional answers. The module creates a severity index for each case, sorting cases automatically for the medical staff.

In some embodiments of the invention, an encoder-decoder is implemented where the submodule suggests further examinations to clarify and validate possible or already established diagnoses.

In some embodiment of the invention, an encoder is implemented where the submodule performs transcriber functions. The transcriber system has as input the audio recording of the medical staff-patient interaction and the audio is transcribed into text format. The transcribed text is supervised medically to ensure medical precision.

In some embodiment of the invention, an encoder is a sound recording and analyzing device. This encoder analyzes the speech of the patient, providing sentiment analysis, comparative analysis with previous recordings, etc.

In some embodiment of the invention, an encoder is an interface for obtaining general information from the national database.

In some embodiment of the invention, an encoder is a video analyzer, that analyzes and determines the physical status of the patient based on the video recording.

In some embodiment of the invention, an encoder is a video analyzer, that analyzes and determines the movement patterns and posture of the patient. The encoder not only outputs the possible pre-diagnose suggestions based on this information alone but outputs the latent information as well. The latent information consists of body landmarks projected onto the patient, showing any discrepancy in the movement patterns and/OR posture. This encoder enables the detection and quantification of different movement disorders and skeletal deformations.

In some embodiment of the invention, an encoder captures images of the physicians notes and digitalizes then tokenizes them.

In some embodiments of the invention, an application-based user interface is deployed for interaction between the patient and the system. The application runs on popular operating systems (such as Windows, Linux and/OR MacOS). The application connects to the local server through a wired or wireless connection.

In some embodiments of the invention, a web-based interface is deployed as the user interface, for interaction between the patient and the system. The web-based interface runs in a browser on a preferred device, connecting securely to a local server.

In some embodiments of the invention, a physical interface is deployed as the user interface, for interaction between the patient and the system. The interaction happens through physical interaction with a device.

In some embodiments of the invention, the central submodule will communicate through tokenized information with an external API, where 3rd-party solutions can be reached.

In some embodiments of the invention, a decoder outputs a bed occupancy strategy that organizes and optimizes bed occupancy.

### Hospitalization Module

In some embodiments of the invention, encoders process data from ultrasound imagery. The ultrasound imagery data can be of 2D and/OR 3D format. The ultrasound imagery can be of of chest and/OR carotid and/OR abdomen and/OR vein and/OR renal and/OR hepatic and/OR cardiac.

In some embodiments of the invention, encoders process CT imagery data. The CT data can be of a whole-body scan and/OR certain regions. The CT data during processing can be segmented and/OR classified and/OR encoded for further processing.

In some embodiments of the invention, encoders process MRI imagery data. The MRI data can be of a whole-body scan and/OR certain regions. The MRI data during processing can be segmented and/OR classified and/OR encoded for further processing.

In some embodiments of the invention, encoders are implemented, where the different types of image-based clinical data (ultrasound, CT and/OR MRI, etc) would be overlapped and concatenated for a more comprehensive depiction of the target part.

In some embodiments of the invention encoders tokenize the different clinical examination data to be processed by LLMs in further processes.

In some embodiments of the invention, encoders suggest different recommended treatments based on patient historical data and current clinical data.

In some embodiment of the invention, an encoder keeps track and analyzes the movement of the patient during hospitalization using an internal tracking system, that can be based on GPS or other technology.

In some embodiments of the invention, the central submodule is based on LLM architecture, which has as input and output tokenized single-/or multimodal data.

In some embodiments of the invention, the outputs of the decoders are visualized on a screen for supervision by medical professionals. After supervision, the output data can be used to control the effectors if any.

In some embodiments of the invention, decoders highlight using segmentation, the pathological or suspicious parts of the imagery for the examiner.

In some embodiments of the invention, decoders will output the translation of the patient's or physician's spoken and/OR written words.

In some embodiment of the invention video streams from cameras inside the operating room are encoded for the central multi-modal submodule.

In some embodiment of the invention analysis of the video streams from the operating room are analyzed, outputting patient position and medical staff movement during surgery.

In some embodiment of the invention, the encoding of video streams from endoscopes and/OR other medical devices is implemented.

In some embodiment of the invention, physiological data is processed and analyzed using machine learning models to monitor and describe the patient's health and reactions and to later compare to available literature and predict the patient future health status.

In some embodiment of the invention, the summarization of surgical procedures is implemented in an encoder submodel.

In some embodiment of the invention, an encoder module integrates the pre-hospitalization data, all the results of the preoperative submodule, and the literature (guidelines, statistics, etc) of the given surgical intervention.

In some embodiment of the invention, an encoder would analyze the video recording of the operation room, segmenting the video images and tracking the movements of surgical staff.

In some embodiment of the invention, an encoder would analyze the POV video stream of the surgeon, segmenting the particular image and labeling anatomical structures, meanwhile keeping track of the stages of the operation according to the guidelines determined previously.

In some embodiment of the invention, an encoder would analyze the intraoperative EEG and CT, and give real-time feedback to the surgical staff.

In some embodiment of the invention, an encoder analyzes the microscopic images of the operated tissue.

In some embodiment of the invention, an encoder detects and locates one or more tissue-implantable device (e.g. ECoG electrode array, deep brain stimulator electrode, neural microelectrode array, heart pacemaker) based on e.g. microscopic images, intra- or post-operative CT, intra- or post-operative MRI images, or a combination of these.

In some embodiments of the invention, an encoder detects the contrastive material and segments the surrounding tissue, generating segmentation and/OR classification and/OR a compressed version of the input data.

In some embodiment of the invention, a decoder suggests areas of the brain for resection as treatment for epilepsy.

In some embodiment of the invention, a decoder suggests a pulse pattern for the treatment of epilepsy through electrode stimulation.

In some embodiment of the invention, a decoder suggests an electrode pathway for inserting electrodes into the brain for deep-brain stimulation, being part of the treatment for Parkinson's disease.

In some embodiment of the invention, a decoder suggests a pulse pattern for increasing the efficiency of deep-brain stimulation in the treatment of Parkinson's disease.

In some embodiment of the invention, a decoder suggests resection strategies for the removal of cancerous tissue.

In some embodiment of the invention, a decoder creates a comprehensive report of the surgical intervention by taking information from different phases, selecting important information from the surgical phase of the hospitalization creating a text-based report accompanied with visual information.

In some embodiment of the invention, a central submodule or decoder would create instructions for surgical digital actuators during operation and the presented strategies would be presented to the supervising medical professionals as suggestions.

### Post-Hospitalization Module

In some embodiment of the invention, an encoder transforms user-input text data into tokens for the central submodule.

In some embodiment of the invention, an encoder transforms user-input health information into tokens to be processed by the central submodule.

In some embodiment of the invention, an encoder searches for relevant studies, summarize them and tokenize the information.

In some embodiment of the invention, an encoder analyzes the captured EMG data from the patient, estimating the current status of recovery, and predicting the speed of recovery in different scenarios.

In some embodiment of the invention, an encoder analyzes the captured EEG data during a certain task, and evaluates different parameters of the brain function.

In some embodiment of the invention, an encoder processes the ECG of the patient during rest and/OR certain tasks and evaluates the cardiovascular performance and rehabilitation of the patient.

In some embodiment of the invention, an encoder processes the motion information of the patient through webcam and/OR infrared cameras.

In some embodiment of the invention, an encoder processes the auto-evaluation of the patient regarding their mood and/OR their perception of the progress of their rehabilitation, etc.

In some embodiment of the invention, an encoder will assess the patient's physiological signs during recovery and warn the patient and/OR medical staff if a bad event is detected or predicted.

In some embodiment of the invention, an encoder will assess the patient's physiological information during recovery and estimate a recovery rate compared to recovery rates with similar conditions in medical studies.

In some embodiment of the invention, an encoder processes pulse-oximeter data during rest and/OR certain tasks.

In some embodiment of the invention, an encoder processes the images and/OR video data, updated by the user about their wound and/OR injury and/OR lesion.

In some embodiment of the invention, an encoder processes the audio data, updated by the user.

In some embodiment of the invention, an encoder processes information about the patient's daily physical activity like number of steps and/OR average length of steps and/OR maximum of steps without rest, etc.

In some embodiment of the invention, an encoder processes patient information and determines if there is an urgency or not.

In some embodiment of the invention, a decoder transforms the output of the central submodule into a human-readable text format. The text format can be presented through a user interface.

In some embodiment of the invention, a decoder transforms the output of the central submodule to an audio format, where the output can be transformed to a well-established audio format. The so generated audio data can be played for the patient, through a third-party speaker.

In some embodiment of the invention, a decoder can present auxiliary data for the patient, facilitating the understanding of the reasons behind post-hospitalization exercises and/OR behavioral alignments.

In some embodiment of the invention, a decoder can generate a personalized nutritional regime for the patient. The personalization can consider the medical history of the patient and/OR the availability of groceries and/OR budgetary aspects and/OR personal preferences.

In some embodiment of the invention, a decoder can generate personalized training schedules for the patient. The personalization can consider the medical history of the patient and/OR the availability of training equipment and/OR physical aspects of the patient and/OR performance on previous exercises.

In some embodiment of the invention, a decoder can make a summary of the patient's current status.

In some embodiment of the invention, a decoder can create predictions of different timelines of the rehabilitation based on the current available information, given by the patient and/OR the medical professional.

In some embodiment of the invention, a decoder can deliver the whole clinical information about the patient in a readable and understandable way for the patient, summarizing the findings during clinical examinations and the process of deciding on the chosen treatment.

### DETAILED DESCRIPTION OF THE INVENTION

During the visit to a medical center, the quality of the care is highly influenced by the medical staff's experience, the administration's effectiveness, and the fluency of information exchange between medical staff and patients. With the presented invention, all three criteria can be improved largely. The presented system complements the different levels of experience of physicians, automatizing the administration and offering a platform for both medical staff and patients where additional information can be obtained. The system architecture is illustrated in FIG. 1, which depicts the flow of data through the multi-modal AI framework. Encoders (101) process raw patient data from various sources and transmit outputs to the pre-hospitalization module (102). The pre-hospitalization module (102) connects to pre-hospitalization decoders (105), which generate user-friendly outputs for initial clinical assessments, while also feeding data into the hospitalization module (103). The hospitalization module (103) receives additional inputs from hospitalization encoders (101), which process real-time clinical data during. The post-hospitalization module (104) also receives input from encoders (101) and the hospitalization module (103), while feeds data to a set of post-hospitalization decoders (105).

As shown in FIG. 2. the information flow is a continuous, starting from different data recorders (206) that are processed to a common latent space by modality-specific encoders (101). The information goes through the central submodule (209) while being supervised by different automatic and/OR semi-automatic and/OR manual supervision (208). The central submodule outputs latent variables to decoders (105) that convert the information from the latent space to modality-specific information, having it as input to the different effectors (207). Before attending the hospital, potential patients can use the public part of the invention. The process of information flow is detailed further in FIG. 3. Users of the public platform of the system can communicate their symptoms through text to a submodule that gives information on potential diagnoses for the provided symptoms (310). Upon the arrival of the patient at the medical center, the patient has to identify themself. The identification can be done in multiple ways, traditionally, by a person with the necessary authority, with biometric authentication using secure hardware modules (e.g., fingerprint recognition with anti-spoofing sensors, facial liveness detection via 3D depth mapping, or iris scanning with motion analysis) to prevent unauthorized access. After the confirmation of the identification process, the electronic health record of the patient is downloaded and/OR accessed (312) from an external database. A subsystem gathers information about the current complaints of the patient (elements of 314, 315, 316, 317 from FIG. 3). The physician recommendation submodule uses a weighted scoring algorithm that integrates physician availability from real-time scheduling APIs, historical patient-physician compatibility scores derived from EHR data, and specialty alignment metrics calculated via natural language processing (NLP) of medical records. Weights are dynamically adjusted based on medical center policies stored in a secure configuration database. The patient has the option to choose from the recommended ones or choose other physicians as well if the preferred one is not on the recommended list. As a third option, the patient will have a physician automatically assigned to them. At the point of the selection of the treating physician, the physician is notified through an app-based notification. In the physician recommendation/assigning process, the subsystem considers multiple parameters, like prior interaction, prior experience of the patient, availability, etc.

During the anamnesis, the physician is supported by several submodules of the system that, for example, alleviate the physician from the burden of administration. Several subsystems record and analyze different aspects of the patient's health and filter different manifestations of possible pathologies using an interface for data collection (311). During the first encounter, the results of these analyses are presented in a human-readable and interpretable format for the physician in real-time, and several suggestions are made, which are presented together with probabilistic confidence metrics generated by a machine learning model trained on historical diagnostic accuracy datasets. The confidence score is calculated as the softmax probability distribution over possible diagnoses, normalized to reflect clinical relevance and prior patient data. The physician can then take action and further examine different symptoms or groups of symptoms to confirm or to rule out certain diagnosis probabilities. The findings of the physician are recorded automatically by the system through a set of data recorders (106) through different subsystems and/OR manually. In both cases the physician supervises the flow of information (108), making modifications and/OR removing information that is incorrectly recorded by the system itself. Every new information that the physician confirms is integrated into the system, potentially considering this new information for the next phases as well (109). Based on the gathered information the possible diagnoses are updated with the confidence scores as well. Other subsystems are updating in real-time the suggested clinical examinations (105 and 106). The suggestions are already limited to the possibilities of the current clinical center, taking into consideration that patient transfer would be necessary to realize a particular examination that has no equipment support in the current center. Separate subsystems will handle the different types of clinical examinations. The different results will be aggregated and through an interface with further suggestions, they will be presented to the physician. The diagnosis is determined by the physician. Based on the accumulated data and the chosen diagnosis, treatments are suggested to the physician. Based on the chosen treatment form, different submodules of the system will continue to assist both the patient to receive as much and as comprehensive information about their condition as possible, and both the physician to present detailed information about the best options on a per-patient basis.

In the case of surgical treatment, the system presented by this invention also supports the procedures in the operating room in several ways, not limited to the following enumeration: audio recording and analysis, automatic evaluation of patient status (ECG, SpO2, ventilation parameters, etc.), suggestions for surgical approaches, automatic surgery documentation etc. A submodule can be an expert in integrating information from the operating room, analyzing and comparing them to recent guidelines, enhancing transparency inside the operating room, and delivering very specific suggestions to medical professionals.

At the end of the hospitalization, a third module of the system offers the possibility of a detailed follow-up of the patients for the physician and offers the patients a platform to personalize the possible rehabilitation routine and/OR keep track of different aspects of the rehabilitation process. Using the submodules of this phase (module), the patients can communicate with an automatic system about their health conditions, where the system gives personalized information back to the patient. One of the submodules of this phase (module) can be an expert at summarizing all the clinical data of the patient, comparing it to similar cases, and suggesting efficient post-hospitalization methods for efficient recovery.

Multi-modal models, including encoder-decoder architectures for text-video fusion and transformer-based cross-modal alignment networks, are pre-trained on deidentified datasets containing 1) audio/speech data from doctor-patient conversations (processed via speech-to-text pipelines), 2) biomedical images (e.g., CT/MRI scans segmented using U-Net models), and 3) clinical notes annotated with ICD-10 codes. Training employs a hybrid approach: initial pre-training on public datasets followed by domain-specific fine-tuning using federated learning across encrypted, decentralized nodes to preserve patient privacy. The datasets are filtered to detect and eliminate low-quality data from the datasets. These datasets are also filtered, to eliminate any information that could lead to the identification of a subject or patient using the data. The training, validation, and test datasets are constructed in a way to eliminate any unnecessary bias, that could introduce false correlations into the trained model. The training data consists of multiple types of data: audio/speech data, image/video data, text data, and electrophysiological data, etc. Audio data consists of general speech data, doctor-patient conversations, intraoperative conversations, etc. Image data consists of images of patients, and images of different biomedical images, such as CT, MRI, X-ray, mammography, etc. Video data consists of video recordings of surgical interventions, patient-doctor interactions, patient movements, etc. Text data consists of texts of medical conversations, medical literature, medical papers, medical guidelines, descriptions of different pathologies, etc. Electrophysiological data consists of different sources of data: electroencephalography (EEG), electromyography (EMG), electrocorticography (ECoG), intracortical electrography (iEEG), electrocardiogramaCG), electrodermal activity (EDA), magnetoencephalography (MEG), electrooculography (EOG), etc.

Single-modality encoders are trained using multi-task learning to project input data into a shared latent representation space aligned via contrastive loss functions. This ensures that features extracted from audio, text, and image modalities are semantically compatible for downstream tasks such as diagnosis prediction.

The models residing in the encoders, the decoders, and the central submodules, can be fine-tuned on the data (medical decisions, corrections by supervision). The fine-tuning of the local models would align the general models to the specifics of the particular medical center. For distributing the experience and intrinsic knowledge of the models, federated learning can be applied on a national level. The federated learning framework uses secure aggregation protocols (e.g., encrypted gradient averaging) to train global models across decentralized medical centers. Local submodels at each center process deidentified data using differential privacy techniques, and model updates are transmitted via HTTPS with TLS 1.3 encryption. A central server computes the mean of local gradients while ensuring no raw patient data is shared between nodes. In the case of transcription modules, LLM, and other modules, data can be shared to enlarge the national database, on which further training can be made. Data with different levels of sensitivity can be distributed differently throughout the system. The shared data that involves personal information that could potentially identify patients can be handled in two possible ways: the data can be preprocessed to a degree that identification could not be established from the so-generated data, or locally running models can be used to process the information.

Because new data is generated on a day-by-day basis, fine-tuning is considered on several different levels:

Local fine-tuning per patient. Sub-modules can be fine-tuned using patients' and/OR medical professionals' input data, together with manual revisions to further fine-tune models to fit certain cases. These cases would contain the most personal information-rich data in their input data to the models. These models can be saved in a local model repository of the medical center where they are managed securely, and locally.

Local fine-tuning per medical center. Sub-modules can be fine-tuned using patients and/OR medical professionals' input data, together with manual revisions to further fine-tune models to fit certain preferences of a medical center. These fine-tunings are managed locally by the medical center. For fine-tuning the modules, deidentification procedures would be applied, maintaining the generalizability of models and avoiding personal information leakage. Global fine-tuning will collect manual revised data from different module inputs and outputs, using federated learning to further fine-tune different submodules on a global scale. Federated learning is implemented using encrypted parameter updates aggregated via a central server. Local models at each medical center compute gradients from de-identified patient data and send these gradients to the global model repository, where they are securely averaged using differential privacy techniques to prevent re-identification of contributing data. Several changes can be streamed to the national model repository, from different medical centers, where these changes are aligned in a certain way (for example the mean of the changes is calculated, the changes are weighted by the number of trained samples, etc.) and the global submodel is updated accordingly, creating a new version of the model. The new model is validated and after certain preconfigured tests have been done to ensure the level of required quality is still met, it will be deployed afterward to the medical centers.

Logging of the system can be implemented on multiple levels to ensure the trackability of the different processes. It is crucial for detecting and resolving any issue that can come with new models, procedures, new guidelines, new integrated hardware, etc. With the logging system, the system supervisors can detect subsystem failures and isolate the faulty subsystem from the rest of the system, optionally deploying a previous fail-safe version of that subsystem, until debugging is done on the current one.

An encoder can have as a function to analyze the outputs of other decoders that are outputting directly or indirectly to the user interface, and classify them—in case of text data—as harmful or harmless data. Different encoders can be deployed for different modalities. The function of these encoders is to ensure that no toxic and harmful data is generated toward the patients. In case of such detection, the data, the metadata of the models is logged, and presented to a system-supervisor. The system supervisor can decide whether the content, identified as harmful, is indeed harmful or not. In case it is not, certain parts of the deidentified data will be stored to fine-tune the encoder for maximizing performance. In case harmful content is correctly identified, the deidentified data is saved to fine-tune the decoder—or the central submodule. Further measures can also be applied based on the severity of the detected content.

### Pre-Hospitalization Module Description

In the phase of patient registration, a physical interface can help obtain certain personal information, based on what a request would go to the national health database, requesting patient history records. The medically non-relevant personal information will be discarded from now on from the pipeline of the system where the encoding/decodings will be effectuated.

The physical interface for patient registration can contain physical buttons and/OR a touch-sensitive screen and/OR audiovisual guidance. The initial patient registration can be performed by the patient, his/her relatives, or the medical staff. In the case of self-registration, the physical interface can contain some validation mechanisms as well, like biometric identification.

The current symptoms can be registered manually, semi-automatically, or automatically. In case of manual registration, the medical staff will write down the symptoms on the ‘superviser interface’. In the case of semi-automatic and automatic registration modes, the system can use encoders to determine the symptoms: from visual, audio cues, and the description of the problem by the patient him (her) self.

The system can transcribe the spoken description of the symptoms by the patient. In semi-supervised mode, the transcription is monitored and corrected if necessary by medical staff.

The encoder from video data employs a convolutional neural network (CNN) trained on datasets containing annotated examples of patient posture, facial expressions, and gait patterns. The CNN processes real-time video input to quantify composure (e.g., via facial expression analysis), detect anomalies in natural movement (e.g., tremors or unsteady gait), and analyze fine-motor coordination (e.g., hand-eye synchronization) using pre-defined thresholds for balance assessment.

Encoder from the audio data can determine the properties of the voice, can detect respiratory symptoms (such as coughs) and encode for the central submodule.

This submodule can provide real-time visualization/live updates of the outputs of the different encoders enabling the live correction of misrepresented data by both parties. In the remote semi-automatic mode, the encoder outputs and their summary is provided to the medical professional staff through a secure network.

The central submodule can generate a description of the patient anamnesis: the document can contain elements from the encoders, like the symptoms communicated by the patient, the results of the objective examination, the relevant history records of the patient, further recommended examinations, and possible diagnosis. This output can be modified by the medical professionals, to minimize any mistakes. In the onsite semi-automatic mode, the medical staff is beside the patient and directly communicates with the patient. In this case, the central submodule only provides suggestions for decoder inputs but certain decoders can be disabled by default.

### Hospitalization Module Description

In some embodiments, during the hospitalization phase of the patient, the system uses the processed information from the pre-hospitalization phase. The hospitalization module can contain several encoders and decoders for analyzing and generating suggestions for treatment and surgical parameters.

Encoders will be used to analyze the outputs of different clinical examinations. The different modalities of data coming from clinical examinations will require several encoders.

The system communicates with the medical center's database, through an Application Programming Interface (API), where the new examination records can be accessed shortly after the examination itself. The appropriate encoder(s) process and analyze the incoming information, generating a processed output. This output can be of many kinds: segmentations, binary output, etc. The outputs of the encoders can trigger, after supervision, the central submodule of the hospitalization module, to regenerate its output and/Or generate additional outputs.

Medical professionals supervise the outputs of some of the encoders before inputting the processed information to the central submodule. A user interface will prompt the results with a full/partial representation of the raw data as well. The user interface is accessible by the designated medical professionals. The communication of the user interface with the central database is performed in a secure way, as well as the access to the user interface.

Every modification on the output data during supervision is noted and the difference is stored and used for model enhancement in the future. The level of fine-tuning (per patient, medical center-wide, or global) will be decided by the medical professional and patient.

The central submodel for the hospitalization model updates the output based on the incoming new encoded information. The part or all of the output of the submodel is connected to the input layers of decoder submodels.

The hospitalization model has a main user interface where the medical staff can view all the information processed in this and in the previous phase in a summarized way: health records can be accessed and viewed, where different visualizations will be available: in case of MRI images, different segmentation results can be toggled. At this stage, the output of the encoders can not be manipulated, but only sent back as feedback for reevaluation with the associated comments and insights.

The main user interface for this module shows the most probable diagnosis in descending order. The list is generated based directly/indirectly on the outputs of the central submodel. The confidence value is shown together with the diagnosis. The user interface can pinpoint certain aspects of the incoming information based on what certain diagnoses are estimated.

After the decision made by the medical staff, the possible treatments and further suggestions are generated by the central submodule. The guidelines and research data for the particular diagnosis are loaded into the system and together with the patient information a personalized treatment plan is suggested. Static supervision at this point is also taken into consideration, where the lack of availability of certain treatment options or medications will induce a personalization of the treatment plan taking into consideration not only the guidelines, research data, and patient data, but the medical center possibilities as well.

The guidelines and research data are also loaded into the system if the chosen treatment consists of surgical procedure phases. If the particular guideline or research data is not available locally, national and/OR international official sources are used to obtain the necessary information.

The decoders of the hospitalization module can generate surgical strategies, taking into consideration the official guidelines, the particularities of the medical center, and the personal health records of the patient.

During surgery, a plethora of sensors will help align the suggestions to the current situation and accurately log the surgical procedures. The video encoders are observing the position and composure of the medical staff as well as the patient in certain cases.

In the case of respective surgeries, a decoder model could generate suggestions on resection strategies. In the case of cerebral tumor resectomy, a decoder model could suggest strategies based on neuroanatomy, and neurovascular knowledge combined with the patient's health records, to offer a personalized treatment, minimalizing invasiveness and maximizing recovery rate.

During respective surgeries, a decoder model employs real-time 3D convolutional neural networks (CNNs) to process intraoperative MRI/CT scans and overlay segmented neuroanatomical landmarks onto augmented reality (AR) displays. The CNN is trained on annotated surgical datasets containing pre- and post-operative imaging pairs, with inference latency optimized using tensorRT for GPU acceleration. Decoders would help in guidance in interventional radiology, etc.

A physical device can be given to the relatives of the patient, where a decoder model would classify the current process of the surgery. The classification would be a binary one, where one option can be “OK”-meaning everything is according to the plans, while the other option can be “ALT”-meaning that there has been an alteration in the strategy or complications have risen.

If any non-standard approaches were used, in the form of a Q&A between medical doctors and the system, these approaches will be documented and integrated into the intervention summary.

The decoders of the intraoperative phase can estimate the important and potentially critical timespans, providing shortcuts and/OR markers to specific timesteps of the operation.

### Post-Hospitalization Module

The post-hospitalization module enables an immediate platform for the patient or the patient's relatives to interact directly with the system to obtain additional information regarding the patient's clinical diagnosis and useful research material for enhancing recovery.

The patient can have access to the post-hospitalization module from inside the medical center through various interfaces: web interface, application-based user interface or physical device-based user interface. During the ending period of the patient's hospitalization period, the patient will be given secured access to the post-hospitalization module of that particular medical center.

Each registered patient has a web-based username and password with additional security measures to protect sensitive data. The interface, which can be accessed outside of medical centers will contain information about the user and the medical centers the patient visited earlier. The recently visited medical center, with an active post-hospitalization module, can be accessed by the user, where several submodules will be available for him/her. The availability of the submodules is determined by the medical staff and confirmed by system supervisors to ensure specific submodules are available and those submodules have the central computational background available to be used efficiently.

The information can be collected through different sensors and interfaces using the post-hospitalization module. Patient or their caretakers will provide different information about the patient's well-being and health status through different interfaces that can have different input modes like checkboxes, text input, classification modes, speech input, audio or visual inputs, etc. The raw information can be preprocessed and encoded locally, remotely, or in a combined way, depending on the processing power of the peripheral device and/OR the sensitivity of the information. The encoded data is then processed remotely, through a central submodule and different decoders, and generated information (summary, possible complications, prediction of rehabilitation, etc) is presented to the medical professional.

The patient, in the form of a Q&A platform, can communicate with the software, that will answer his/her questions based on the summary created earlier. The post-hospitalization Q&A platform uses a fine-tuned large language model (LLM) with domain-specific knowledge distillation from PubMed-indexed medical literature and institutional guidelines. The LLM is hosted in a HIPAA-compliant cloud environment, where queries are processed via an API that redacts patient identifiers using regular expression matching before inputting text to the model. With this information, the Q&A platform will be able to provide high-quality information and lifestyle advice in a personalized way.

If the user interface provided platform is not satisfactory for answering specific questions, the patient can request a direct consultation with the attending physician through the system. This option will be available based on the settings set up by the medical professional.

The medical professional can also request the patient through the platform, to attend the medical center for further examinations.

The system provides, through the patient-side platform an opportunity for supervision by their care-takers. The platform offers a special user-type registration, where the caretakers can join the system and they can be added as supervisors for the patient. This is done after authentication and confirmation by the patient. On this special platform, the caretakers can supervise the completion of the medication and/OR rehabilitation tasks. Through this same system caretakers are notified when an urgent or critical status is detected.

The platform automatically calls for first responders in case of detected urgency. In such cases, the decoder submodules generate a summary of the patient's information to help with proper treatment. The platform also notifies the attending physician if available and with decode submodules provides vital information about the patient.

Medical professionals can manipulate the post-hospitalization phase, being able to shorten, expand, or terminate the phase. In case of such modification, the patient gets a notification about the modification and a text with supporting comments on the decision from the medical professional. In case no modification is performed, the post-hospitalization support will continue until it was established initially.

### BRIEF DESCRIPTION OF THE DRAWINGS

FIG. 1. is a block diagram of the general information flow of the invention

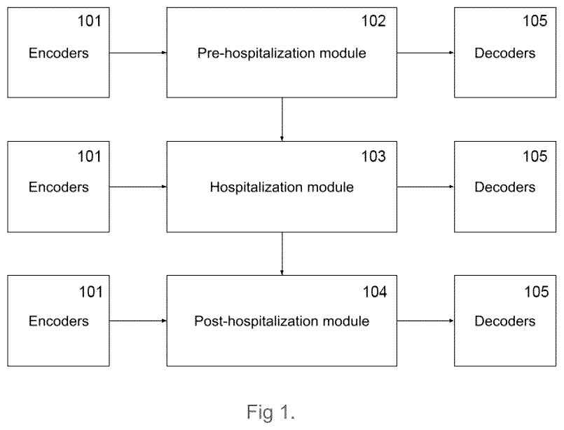

FIG. 2. is a block diagram showing different encoding and decoding structure along with supervision in some of the embodiments of the invention

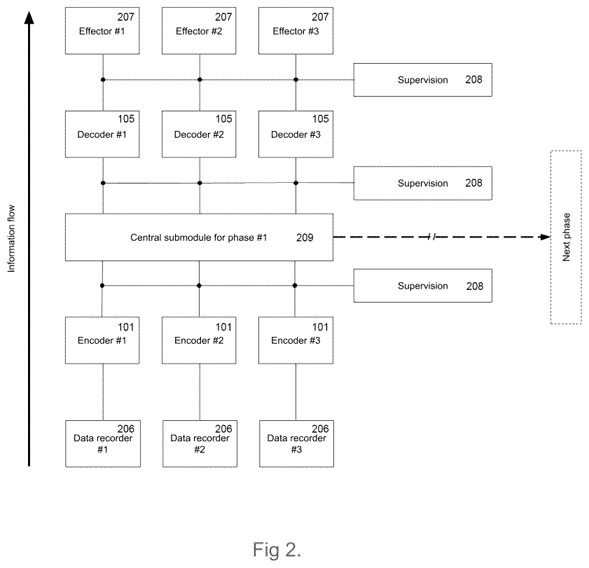

FIG. 3. is a block diagram showing the different stages of a possible embodiment of the invention during pre-hospitalization phase

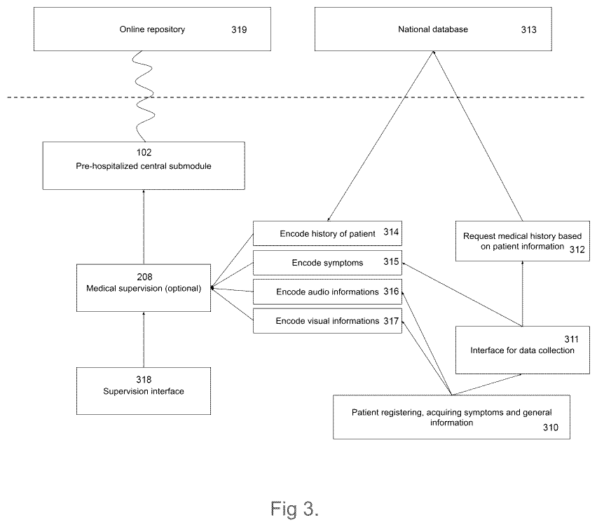

What is claimed is:

1. A system for generating personalized medical information, comprising:
a. a plurality of encoder submodules configured to process multi-modal patient data from different stages of hospitalization into encoded representations in a latent space;
b. a central submodule operatively connected to the encoders and trained on medical datasets using machine learning/deep learning methods, including Large Language Models (LLMs), for integrating said encoded representations into a unified representation; and
C. a plurality of decoder submodules operatively connected to the central submodule, configured to transform outputs from the latent space into user-friendly modalities, wherein the system is structured to provide real-time feedback during hospitalization phases, enable dynamic supervision by medical professionals, and generate personalized clinical decisions for patients.

2. The system of claim 1, wherein the central submodule is trained using federated learning across multiple medical centers, enabling decentralized model updates while preserving patient data privacy through deidentification protocols.

3. The system of claim 1 or 2, further comprising a pre-hospitalization module configured to collect and process patient data prior to admission, including:
a. an encoder submodule for transcribing audio recordings of physician-patient consultations;
b. an encoder submodule for analyzing video data to detect posture, movement patterns, and skeletal deformities; and
c. a decoder submodule for generating diagnosis suggestions with confidence scores based on encoded data.

4. The system of claim 1 or 2, further comprising a hospitalization module including submodules for processing medical imaging data and surgical guidance, wherein the module includes:
a. encoders configured to process ultrasound, CT, and MRI imagery in 2D/3D formats;
b. decoders configured to suggest resection strategies for cerebral tumors based on neuroanatomy and patient-specific health records; and
c. a central submodule trained on neurovascular knowledge and surgical guidelines to align with real-time intraoperative data.

5. The system of claim 1 or 2, further comprising a post-hospitalization module configured to monitor recovery, provide rehabilitation guidance, and update medical records, including:
a. decoders configured to generate personalized rehabilitation plans using machine learning algorithms;
b. an emergency alert system that automatically notifies medical personnel in case of abnormal vital signs or self-reported symptoms; and
c. a medication management tool that tracks prescribed medications and provides reminders for dosing times.

6. The system of claim 1, wherein the central submodule includes dynamic supervision mechanisms allowing medical professionals to adjust system outputs based on patient needs, including:
a. semi-automatic modes with remote or onsite interactivity; and
b. logging capabilities to track modifications made by supervisors for model enhancement.

7. The system of claim 1, further comprising a secure network infrastructure for real-time data transmission between modules, including:
a. encrypted communication channels for data transfer;
b. access control protocols to restrict unauthorized use; and
c. redundancy mechanisms to ensure continuous operation during critical procedures.

8. The system of claim 1, wherein the central submodule is configured to integrate data from multiple encoders operating in parallel, dynamically allocating computational resources based on the complexity of incoming data streams and prioritizing critical information for real-time decision-making by medical professionals.

9. The system of claim 5, wherein the post-hospitalization module includes a personalized rehabilitation planner that utilizes patient-specific data from pre-hospitalization, hospitalization, and post-hospitalization phases to generate exercise routines tailored to the patient's condition, using machine learning algorithms to predict optimal intensity and duration.

10. The system of claim 5, wherein the emergency alert system is integrated with decoder submodules to automatically notify medical personnel in case of detected urgency, including:
a. generation of a summary of the patient's information for proper treatment; and
b. notification of the attending physician if available, with vital information provided via decoded outputs.

11. The system of claim 1 or 2, further comprising a hospitalization module including at least one submodule for processing data yielded by neural electrodes.
